(19) United States
(12) Patent Application Publication
(10) Pub. No.: US 2009/0110718 A1
(43) Pub. Date: Apr. 30, 2009

(54) Derivatives of tiacumicin B as anti-cancer agents

(75) Inventors: Mei-Chiao Wu, Zhongli City (TW); Chang-Cheng Huang, Keelung City (TW); Yi-Chen Lu, Judong Town (TW); Wei-Jen Fan, Taipei City (TW)

Correspondence Address: BACON & THOMAS, PLLC, 625 SLATERS LANE, FOURTH FLOOR, ALEXANDRIA, VA, 22314-1176, US

(73) Assignee: Echem Hightech Co., Ltd., Hsin-Chu Hsien (TW)

(21) Appl. No.: 12/071,625

(22) Filed: Feb. 25, 2008

(30) Foreign Application Priority Data
Oct. 26, 2007 (TW) 096140405

(51) Int. Cl. A61K 9/70; C07D 313/00; A61K 31/357; A61P 35/04; A61K 9/127; A61K 9/00

(52) U.S. Cl. 424/449; 549/346; 514/450; 424/450; 424/400

(57) ABSTRACT

New derivatives of tiacumicin B are disclosed and are presented by the general formula:

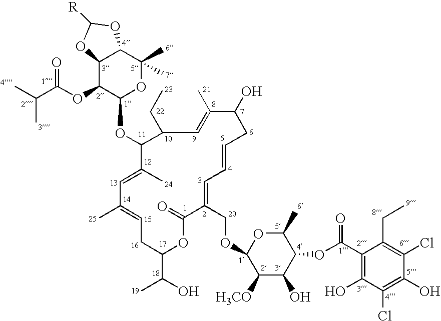

wherein R is

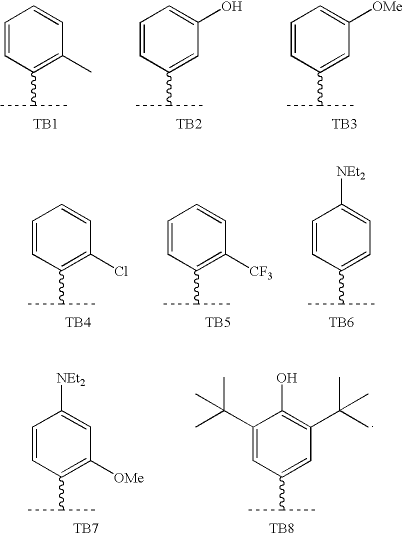

The use of these derivatives as a method for treating cancer or tumor is disclosed. A method of treating cancer or tumor by administrating the patient a composition comprising a therapeutically effective amount of the tiacumicin B is also disclosed.

### FIELD OF THE INVENTION

The present invention is directed to the derivatives of tiacumicin B and to the use of these derivatives for cancer or tumor treatment. Moreover, the present invention is also directed to a method for cancer or tumor treatment by using a known antibiotic, tiacumicin B.

### BACKGROUND OF THE INVENTION

Tiacumicins are the members of a family of 18-membered macrocycles derived from the fermentation of Dactylosporangium aurantiacum subsp. hamdenensis. Several structural isomers have been identified within the tiacumicin family. Tiacumicins have a moderate activity against most gram-positive bacteria, especially tiacumicin B with the excellent activity towards Clostridium, and are bactericidal against these organisms. The chemical structure of Tiacumicin B has been elucidated in the J. of Antibiotics XL(5): 567-574, 575-588 (May 1987) and the detailed manufacturing processes for tiacumicin A, B, C, D, E, F have been explained in U.S. Pat. No. 4,918,174.

Several alkylated tiamumicins are described in U.S. Pat. No. 5,583,115 and some brominated derivatives of tiamumicins are later disclosed in U.S. Pat. No. 5,767,096. Both these tiamumicin derivatives are produced on the purposes of treating the bacterial infections in mammals, just as exactly same as the already known bioactivities in the tiamumicins in U.S. Pat. No. 4,918,174. The bioactivity of all the above-mentioned tiacumicins are expected and proved therefore to act against a number of bacterial pathogens. None of these patents and documents discloses or even suggests the information that there exists the anti-cancer bioactivity in the tiamumicins and their derivatives.

The present invention discloses tiacumicin B and several new derivatives of tiacumicin B exhibiting excellent inhibitory activity against the cell growth in several cancer cell-lines.

### SUMMARY OF THE INVENTION

Tiacumicins has been known to be the novel anti-microbial agents and can be obtained from the fermentation broth and mycelium of Dactylosporangium aurantiacum subsp. Hamdenensis. These tiacumicin antibiotics comprise tiacumicin A to F and can be further chemically modified by bromination and alkylation. The compounds of this invention are the derivatives of tiacumicin B. All of them have similar structures with each other and are found to be effective in vitro in inhibiting the growth of cancer cells.

It is an object of the present invention to provide compounds having the following formula:

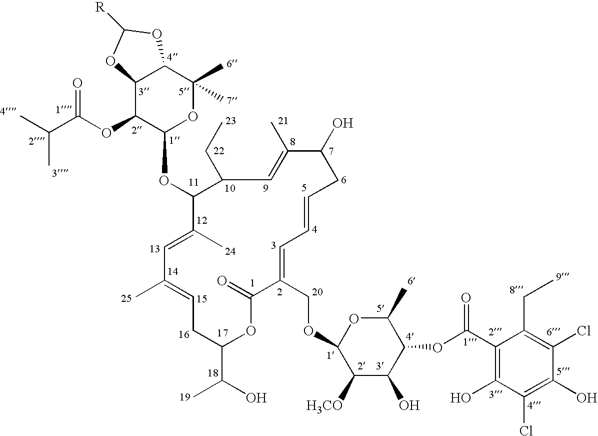

wherein R is selected from the group consisting of 2-methylbenzylidene acetal, 3-hydroxylbenzylidene acetal, 3-methoxylbenzylidene acetal, 2-chlorobenzylidene acetal, 2-trifluoromethylbenzylidene acetal, 4-N,N-diethylaminobenzylidene acetal, 4-N,N-diethylamino-2-methoxybenzylidene acetal and 4,5-tert-dibutyl-4-hydroxybenzylidene acetal, having the following formulas, respectively:

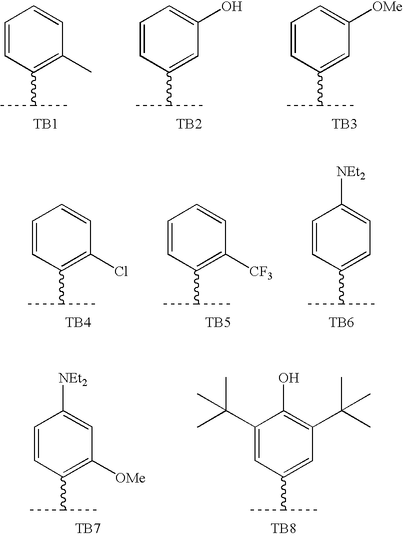

TB1: 2-methylbenzylidene acetal

TB2: 3-hydroxylbenzylidene acetal

TB3: 3-methoxylbenzylidene acetal

TB4: 2-chlorobenzylidene acetal

TB5: 2-trifluoromethylbenzylidene acetal

TB6: 4-N,N-diethylaminobenzylidene acetal

TB7: 4-N,N-diethylamino-2-methoxybenzylidene acetal

TB8: 4,5-tert-dibutyl-4-hydroxybenzylidene acetal

It is another object of the present invention to provide a method of treating cancer or tumor by administrating the patient a composition comprising a therapeutically effective amount of the compound of the present invention.

It is still an object of the present invention to provide a method of treating cancer or tumor by administrating the patient a composition comprising a therapeutically effective amount of the tiacumicin B which has the following formula:

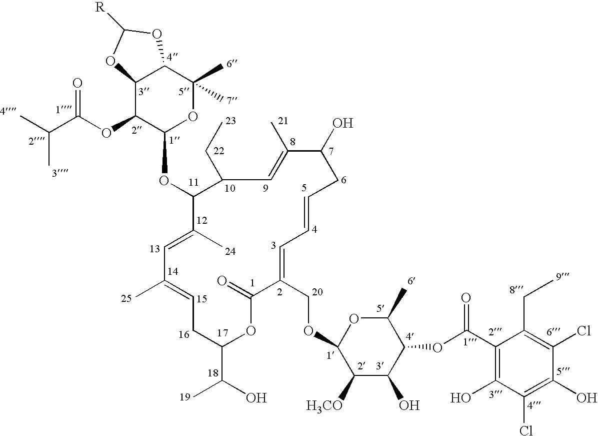

### BRIEF DESCRIPTION OF THE DRAWINGS

FIGS. 1 through 3 are graphs showing the results of treating cancer cells with tiacumicin B, TB1 through TB8, and the positive control, tamoxifen.

FIG. 4 is a graph showing the result of treating normal cells with tiacumicin B, TB1 through TB8, and the positive control, tamoxifen.

### DETAILED DESCRIPTION OF THE PREFERRED EMBODIMENTS

In vitro tests are conducted to establish the activities that each of tiacumicin B and TB1 through TB8 inhibits the growth of the cancer cell-lines. The testing methodologies are described below.

Experiments were performed using different cancer cell-lines, which were supplied by the American Type Culture Collection (ATCC), Rockville, Md., USA, and maintained in the recommended culturing media. The detailed steps and condition for cell culture are described as the following:

### I—Cell-lines:

MCF7, human breast adenocarcinoma: Minimum essential medium (Eagle) with 2 mM L-glutamine and Earle's BSS adjusted to contain 1.5 g/L sodium bicarbonate, 0.1 mM non-essential amino acids and 1.0 mM sodium pyruvate and supplemented with 0.01 mg/ml insulin, 90%; fetal bovine serum, 10%.

T-47D, human breast ductal carcinoma: RPMI 1640 medium with 2 mM L-glutanine adjusted to contain 1.5 g/L sodium bicarbonate, 4.5 g/L glucose, 10 mM HEPES, and 1.0 mM sodium pyruvate and supplemented with 0.01 mg/ml insulin, 90%; fetal bovine serum, 10%.

HeLa, humen cervical adenocarcinoma: Minimum essential medium (Eagle) with 2 mM L-glutamine and Earle's BSS adjusted to contain 1.5 g/L sodium bicarbonate, 0.1 mM non-essential amino acids and 1.0 mM sodium pyruvate and supplemented with 0.01 mg/ml insulin, 90%; fetal bovine serum, 10%.

WS1, human fetal skin fibroblast: Minimum essential medium (Eagle) with 2 mM L-glutamine and Earle's BSS adjusted to contain 1.5 g/L sodium bicarbonate, 0.1 mM non-essential amino acids and 1.0 mM sodium pyruvate and supplemented with 0.01 mg/ml insulin, 90%; fetal bovine serum, 10%

Tamoxifen is obtained from Sigma-Aldrich. All test samples were dissolved in absolute ethanol and serial diluted to the testing concentrations. For blank sample, absoluted ethanol was used. The final organic solvent content for all cell assays were less than or equal to 0.1%.

### II—Cell Viability Assay:

The cell viability assay was conducted by using the method described by J. Carmichael, et al in Cancer Research 47:936 (1987). All the cells were seeded into 96-well microtitre plates and incubated at 37° C. under 5% CO₂ atmosphere. For MCF7, T-47D and WS1, 3000 cells/100 μl/well were used and for HeLa, 1000 cells/100 μl/well were used. After 24 hours of incubation, culturing media were replaced with fresh media containing 2% fetal bovine serum and then treated with different concentrations (1 to 10 μM) of test samples: tamoxifen, tiacumicin B, TB1 through TB8 or left untreated. After additional 72 hours of incubation at 37° C. under 5% CO₂ atmosphere, the cell were then collected and evaluated using MTT assay to assess the cell viability by using the method mentioned above.

### III—Results:

### (1) Breast Cancer Cells

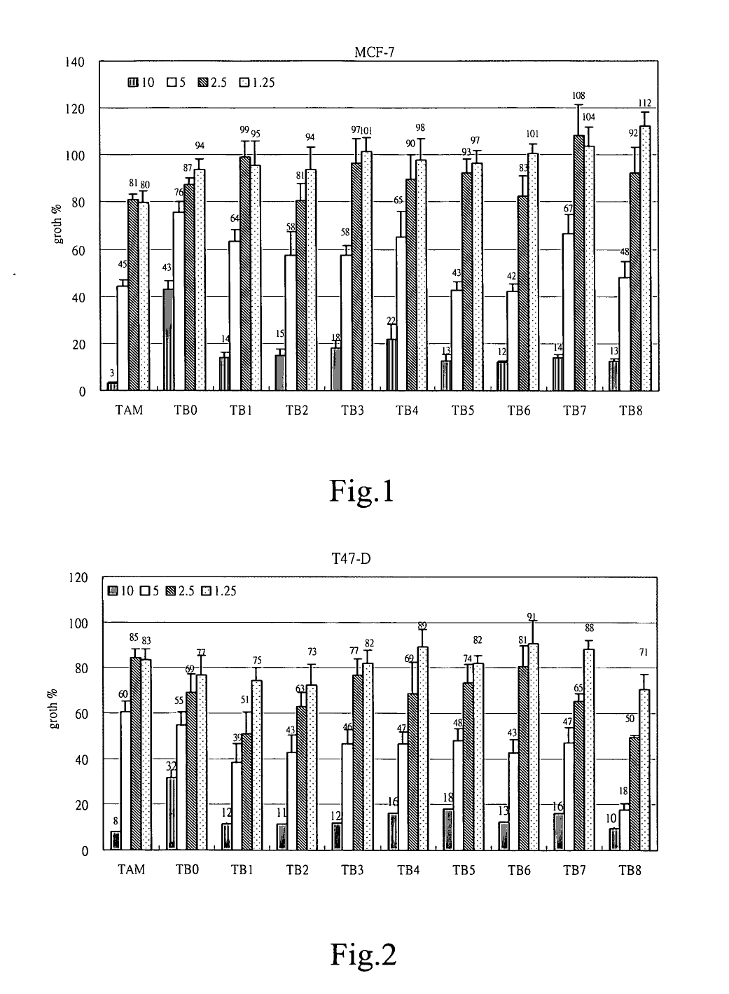

The results of the treatment of human breast cells (MCF7 and T-47D) with tamoxifen (TMA), tiacumicin B (TB) and TB1 to TB8 are shown in FIGS. 1 and 2, respectively. Cultures containing 3000 cells per well were treated with 0, 1, 2.5, 5 and 10 μM (final concentration) of the test samples and the viable cells were counted after 72 hrs. As shown in FIGS. 1 and 2, significant inhibition to the cancer cell growth was observed for tiacumicin B and all of its derivatives at the concentrations tested. In addition, all test samples were cytotoxic at concentration equal to or greater than 10 μM. In both FIG. 1 and FIG. 2, error bars are indicated the estimated standard deviation based on three experiments.

TABLE 1
Observed IC₅₀ for MCF7 breast cancer cells treated with different test
samples

| Test Compound | IC₅₀ value |
|---|---|
| tamoxifen (TMA) | 6.35 ± 0.45 |
| tiacumicin B (TB) | 8.39 ± 1.00 |
| TB1 | 7.06 ± 0.83 |
| TB2 | 6.90 ± 0.36 |
| TB3 | 7.09 ± 0.54 |
| TB4 | 6.81 ± 0.01 |
| TB5 | 5.95 ± 0.46 |
| TB6 | 5.98 ± 0.54 |
| TB7 | 6.19 ± 0.68 |
| TB8 | 5.78 ± 0.81 |

TABLE 2
Observed IC₅₀ for T-47D breast cancer cells treated with different test
samples

| Test Compound | IC₅₀ value |
|---|---|
| tamoxifen (TMA) | 5.50 ± 0.21 |
| tiacumicin B (TB) | 5.56 ± 0.57 |
| TB1 | 3.99 ± 0.58 |
| TB2 | 4.24 ± 0.05 |
| TB3 | 4.99 ± 0.22 |
| TB4 | 5.66 ± 0.62 |
| TB5 | 4.66 ± 0.57 |
| TB6 | 4.77 ± 0.51 |
| TB7 | 4.80 ± 0.46 |
| TB8 | 4.04 ± 1.09 |

### (2) HeLa Cervical Cancer Cells

TABLE 3
Observed IC₅₀ for HeLa cervial cancer cells treated with different test
samples

| Test Compound | IC₅₀ value |
|---|---|
| tamoxifen (TMA) | 6.10 ± 0.53 |
| tiacumicin B (TB) | 8.54 ± 1.03 |
| TB1 | 5.88 ± 0.42 |
| TB2 | 5.98 ± 0.22 |
| TB3 | 5.99 ± 0.45 |
| TB4 | 5.69 ± 0.24 |
| TB5 | 5.60 ± 0.37 |
| TB6 | 5.56 ± 0.13 |
| TB7 | 5.30 ± 0.49 |
| TB8 | 5.76 ± 0.32 |

Table 1 shows the IC₅₀ value for tamoxifen, tiacumicin B and TB1 through TB8 observed in the cell viability assay of MCF7. The most potent compound was TB2 whose IC₅₀ value was superior to that of tamoxifen. The observed IC₅₀ value for T-47D treated with different concentrations (1 to 10 μM) of test samples is shown in Table 2. All TB1 through TB8 were equally potent to that of tanoxifen.

The results of treatment of HeLa human cervical adenocacinoma cells with tiacumicin B and TB1 through TB8 are shown in FIG. 3. Error bars are indicated the estimated standard deviation based on three experiments. Their IC₅₀ values are summarized in Table 3. The most potent compound among the series was TB6 whose IC₅₀ value was superior to that of tanoxifen.

### (3) Toxic Effects of Normal Cells

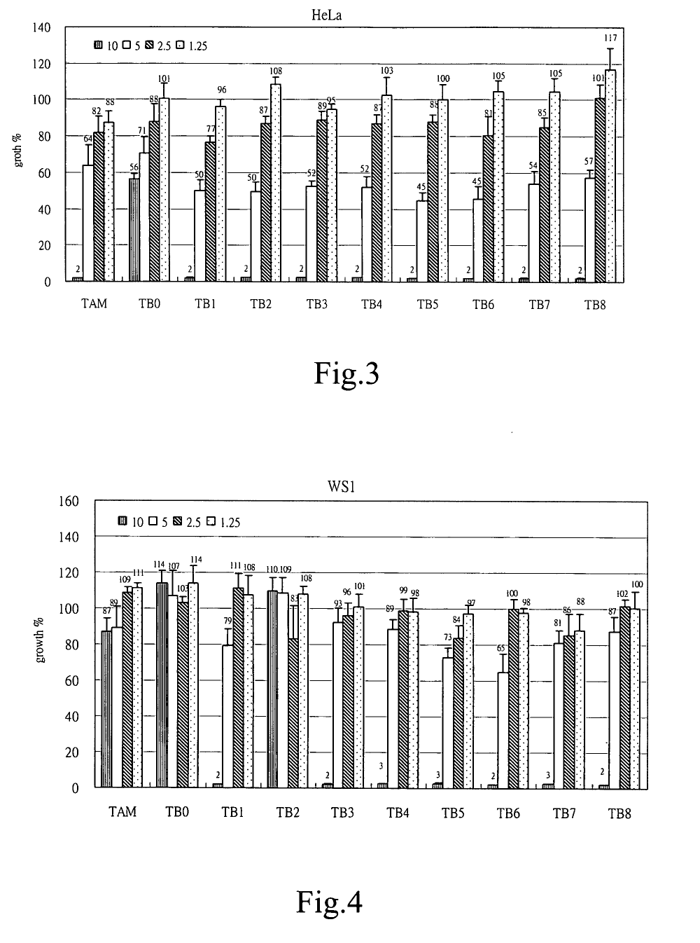

Normal cells (shown in FIG. 4), normal human skin fibroblast WS1 in the concentrations used in the above-described experiments and no significant toxic effects on the cells were noted. Toxic effects begin to show up at about 10 μM in these types of cells.

It is clear from the data, shown in FIG. 1 to 4 and Table 1 to 3, that tiacumicin B and chemical compounds similar to tiacumicin B possess excellent inhibitory activities against human breast or cervical cancer cells. The lack of toxic effects on normal cells at the concentrations needed for effectiveness against the cancer cells shows that tiacumicin B and the chemical compounds of its derivatives are very potent chemotheraputic reagents. These pharmaceutical compositions containing the active agent of the present invention are suitable for administration to humans or other mammals and they are typically produced in a germfree condition. No carcinogen or other toxic or pyrogenic matters which cause an adverse reaction should exist. The pharmaceutical compositions containing the active agent of the present invention can be administrated via several routes, for example by oral, rectal, nasal, topical, transdermal, or parental (including intravenous, intramuscular and subcutaneous) administration. Parental administration can be accomplished by using traditional syringes and needles, or a high pressure injection technique, such as POWDERJCT™.

The compounds and pharmaceutical compositions thereof include those wherein the active ingredient is administrated in an effective amount to achieve the intended purpose. More specifically, a “therapeutically effective amount” means an amount effective to prevent the development of, or to cure, or to alleviate the existing symptoms of, the subject being treated. Determination of a therapeutically effective amount is well known within the capability of the skilled in the art.

### IV-Synthesis of the Benzylidene Acetals of Tiacumicin B:

The synthesis of the benzylidene acetals of tiacumicin B in the present invention are as followed:

TB1: To a solution of tiacumicin B (0.53 g, 0.5 mmole) in dry THF (10 ml) was added 2-methylbenzaldehyde (60 mg, 0.5 mmole), p-toluenesulfonic acid (catalytic amount 10 mg), and anhydrous CuSO₄ (1 g). The solution was stirred at room temperature for several days. After the seventh day, solvent was removed and the crude product was purified on a column. 7:3 hexane/ethyl acetate was used to remove un-reacted benzaldehyde and the major product isolated using 1:1 hexane/EA. After purification, 174 mg (50% yield) of product was isolated. ¹³C-NMR chemical shift assignment for TB1 is summarized in Table 4.

TB2: To a solution of tiacumicin B (0.53 g, 0.5 mmole) in dry THF (10 ml) was added 3-hydroxybenzaldehyde (61 mg, 0.5 mmole), p-toluenesulfonic acid (catalytic amount 10 mg), and anhydrous CuSO₄ (1 g). The solution was stirred at room temperature for several days. After the seventh day, solvent was removed and the crude product was purified on a column. 7:3 hexane/ethyl acetate was used to remove un-reacted benzaldehyde and the major product isolated using 1:1 hexane/EA. After purification, 191 mg (55% yield) of product was isolated. ¹³C-NMR chemical shift assignment for TB2 is summarized in Table 4.

TB3: To a solution of tiacumicin B (0.53 g, 0.5 mmole) in dry THF (10 ml) was added 3-methoxybenzaldehyde (68 mg, 0.5 mmole), p-toluenesulfonic acid (catalytic amount 10 mg), and anhydrous CuSO₄ (1 g). The solution was stirred at room temperature for several days. After the seventh day, solvent was removed and the crude product was purified on a column. 7:3 hexane/ethyl acetate was used to remove un-reacted benzaldehyde and the major product isolated using 1:1 hexane/EA. After purification, 100 mg (28% yield) of product was isolated. ¹³C-NMR chemical shift assignment for TB3 is summarized in Table 4.

TB4: To a solution of tiacumicin B (0.53 g, 0.5 mmole) in dry THF (10 ml) was added 2-chlorobenzaldehyde (70.3 mg, 0.5 mmole), p-toluenesulfonic acid (catalytic amount 10 mg), and anhydrous CuSO₄ (1 g). The solution was stirred at room temperature for several days. After the seventh day, solvent was removed and the crude product was purified on a column. 7:3 hexane/ethyl acetate was used to remove un-reacted benzaldehyde and the major product isolated using 1:1 hexane/EA. After purification, 163 mg (43% yield) of product was isolated. ¹³C-NMR chemical shift assignment for TB4 is summarized in Table 4.

TB5: To a solution of tiacumicin B (0.53 g, 0.5 mmole) in dry THF (10 ml) was added 2-trifluoromethylbenzaldehyde (87 mg, 0.5 mmole), p-toluenesulfonic acid (catalytic amount 10 mg), and anhydrous CuSO₄ (1 g). The solution was stirred at room temperature for several days. After the seventh day, solvent was removed and the crude product was purified on a column. 7:3 hexane/ethyl acetate was used to remove un-reacted benzaldehyde and the major product isolated using 1:1 hexane/EA. After purification, 180 mg (50% yield) of product was isolated. ¹³C-NMR chemical shift assignment for TB5 is summarized in Table 4.

TB6: To a solution of tiacumicin B (0.53 g, 0.5 mmole) in dry THF (10 ml) was added 4-N,N-diethylaminobenzaldehyde (89 mg, 0.5 mmole), p-toluenesulfonic acid (catalytic amount 10 mg), and anhydrous CuSO₄ (1 g). The solution was stirred at room temperature for several days. After the seventh day, solvent was removed and the crude product was purified on a column. 7:3 hexane/ethyl acetate was used to remove un-reacted benzaldehyde and the major product isolated using 1:1 hexane/EA. After purification, 76 mg (21% yield) of product was isolated. ¹³C-NMR chemical shift assignment for TB6 is summarized in Table 4.

TB7: To a solution of tiacumicin B (0.53 g, 0.5 mmole) in dry THF (10 ml) was added 4-N,N-diethylamino-2-methoxybenzaldehyde (104 mg, 0.5 mmole), p-toluenesulfonic acid (catalytic amount 10 mg), and anhydrous CuSO₄ (1 g). The solution was stirred at room temperature for several days. After the seventh day, solvent was removed and the crude product was purified on a column. 7:3 hexane/ethyl acetate was used to remove un-reacted benzaldehyde and the major product isolated using 1:1 hexane/EA. After purification, 134 mg (76% yield) of product was isolated. ¹³C-NMR chemical shift assignment for TB7 is summarized in Table 4.

TB8: To a solution of tiacumicin B (0.53 g, 0.5 mmole) in dry THF (10 ml) was added 3,5-tert-dibutyl-4-hydroxybenzaldehyde (117 mg, 0.5 mmole), p-toluenesulfonic acid (catalytic amount 10 mg), and anhydrous CuSO₄ (1 g). The solution was stirred at room temperature for several days. After the seventh day, solvent was removed and the crude product was purified on a column. 7:3 hexane/ethyl acetate was used to remove un-reacted benzaldehyde and the major product isolated using 1:1 hexane/EA. After purification, 173 mg (45% yield) of product was isolated. ¹³C-NMR chemical shift assignment for TB8 is summarized in Table 4.

TABLE 4

| | | Chemical shift of |
|---|---|---|
| | Chemical shift | Tiacumicin B in reference |
| No. of carbon | (ppm) | (ppm)* |
| 23 | 11.3 | 11.3 |
| 24 | 13.9 | 13.9 |
| 19 | 14.5 | 14.6 |
| 21 | 15.4 | 15.4 |
| 25 | 17.0 | 17.5 |
| 6′ | 18.1 | 18.1 |
| 7″ | 18.7 | 18.2 |
| 3′′′′ | 19.1 | 19.1 |
| 4′′′′ | 19.5 | 19.5 |
| 9′′′ | 20.3 | 20.2 |
| 8′′′ | 26.4 | 26.5 |
| 22 | 26.9 | 26.9 |
| 16 | 28.3 | 28.4 |
| 6″ | 28.7 | 28.7 |
| 2′′′′ | 35.4 | 35.4 |
| 6 | 37.3 | 37.3 |
| 10 | 42.5 | 42.5 |
| 2′-OCH3 | 62.2 | 62.2 |
| 20 | 63.9 | 63.9 |
| 18 | 68.2 | 68.3 |
| 3″ | 70.5 | 70.5 |
| 2′ | 71.6 | 71.6 |
| 7 | 72.7 | 72.8 |
| 3′ | 73.2 | 73.2 |
| 2″ | 73.5 | 73.5 |
| 5″ | 74.5 | 74.5 |
| 4″ | 75.9 | 75.9 |
| 4′ | 76.9 | 76.8 |
| 17 | 78.6 | 78.6 |
| 5′ | 82.4 | 82.5 |
| 11 | 94.3 | 94.3 |
| 1″ | 97.2 | 97.1 |
| 1′ | 102.2 | 102.2 |
| 4′′′ | 108.7 | 108.9 |
| 2′′′ | 112.8 | 110.7 |
| 6′′′ | 114.8 | 115.7 |
| 9 | 124.5 | 124.6 |
| 2 | 125.6 | 125.6 |
| 15 | 126.9 | 126.8 |
| 4 | 128.5 | 128.5 |
| 13 | 134.6 | 134.6 |
| 14 | 136.3 | 136.3 |
| 12 | 137.0 | 136.9 |
| 8 | 137.0 | 137.0 |
| 7′′′ | 141.8 | 141.9 |
| 5 | 143.7 | 143.7 |
| 3 | 146.2 | 146.3 |
| 3′′′ or 5′′′ | 153.8 | 155.4 |
| 3′′′ or 5′′′ | 154.5 | 155.8 |
| 1 | 169.1 | 169.1 |
| 1′′′ | 169.7 | 170.1 |
| 1′′′′ | 178.4 | 178.4 |

Hydroxybenzylidine acetal:

| | |
|---|---|
| CH (acetal) | 114.7 |
| C-5 | 115.1 |
| C-6 | 118.6 |
| C-4 | 120.2 |
| C-2 | 129.8 |
| C-1 | 138.6 |
| C-3 | 157.2 |

*Hochlowski et al. (1987) J. of Antibiotics 40: 575.

The inventive subject matter being thus described, it will be obvious that the same may vary in many ways. Such variations are not to be regarded as a departure from the spirit and scope of the inventive subject matter, and all such modifications are intended to be included within the scope of the following claims.

What is claimed is:

1. A compound having the formula: 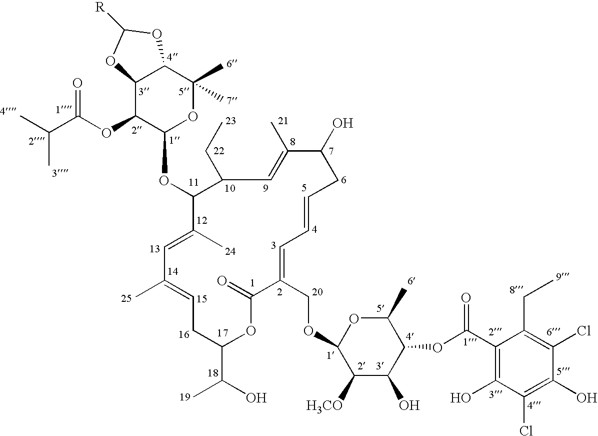
wherein R is:

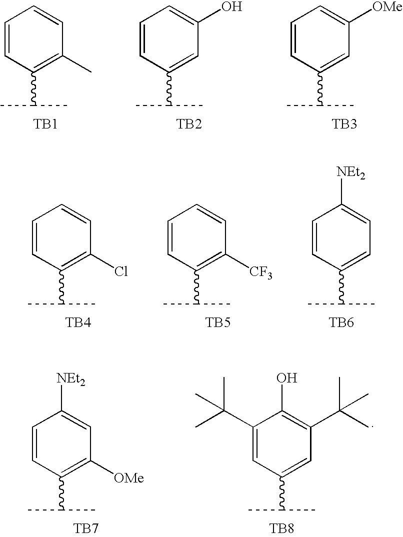

2. A method for treating a tumor or cancer in a patient comprising administrating a composition comprising an effective amount of the compound of claim 1, wherein said tumor or cancer is responsive to said composition.

3. A method of claim 2, wherein said tumor or cancer is present in lung, breast, colon, prostate, pancreas, stomach, liver, brain, kidney, uterus, cervix, ovaries, urinary tract, rectal tract, thyroid gland, bone, esophagus, nasopharynx, or is a melanoma or leukemia.

4. A method of claim 2, wherein said composition is injected directly into the tumor location site or is administrated orally, intravenously, intramuscularly, intradermally, subcutaneously, topically or intravenously in the form of a lipsome, with or without pharmaceutically acceptably carrier.

5. A method of claim 3, wherein said composition is injected directly into the tumor location site or is administrated orally, intravenously, intramuscularly, intradermally, subcutaneously, topically or intravenously in the form of a lipsome, with or without pharmaceutically acceptably carrier.

6. A device for intravenous, intramuscular, intradermal or subcutaneous administration comprising a syringe needle and a syringe;
wherein said syringe needle is connected to said syringe, and
wherein said syringe contains a suspension or solution of an effective amount of compound the compound of claim 1.

7. A device for transdermal administration comprising a patch; wherein said syringe contains a suspension or solution of an effective amount of compound the compound of claim 1.

8. A method for treating a tumor or cancer in a patient comprising administrating a composition comprising an effective amount of the compound of tiacumicin B, wherein said tumor or cancer is responsive to said composition.

9. A method of claim 8, wherein said tumor or cancer is present in lung, breast, colon, prostate, pancreas, stomach, liver, brain, kidney, uterus, cervix, ovaries, urinary tract, rectal tract, thyroid gland, bone, esophagus, nasopharynx, or is a melanoma or leukemia.

10. A method of claim 9, wherein said composition is injected directly into the tumor location site or is administrated orally, intravenously, intramuscularly, intradermally, subcutaneously, topically or intravenously in the form of a lipsome, with or without pharmaceutically acceptably carrier.

11. A process comprises the step of using the tiacumicin B in the manufacture of a medicament for the treatment of tumor or cancer.
